(19) United States
(12) Patent Application Publication
(10) Pub. No.: US 2026/0348944 A1
(43) Pub. Date: Nov. 5, 2026

(54) CELLULAR-BASED METHOD FOR DETERMINING THE POTENCY OF DEFIBROTIDE

(71) Applicant: Gentium S.R.L., Villa Guardia (co) (IT)

(72) Inventors: Terenzio IGNONI, San Fermo Della Battaglia (CO) (IT); Vijay KUMAR, Casnate (IT); Claudio VERGA, Bregnano (CO) (IT)

(21) Appl. No.: 19/547,441

(22) Filed: Feb. 23, 2026

Related U.S. Application Data
(63) Continuation of application No. 18/170,644, filed on Feb. 17, 2023, now Pat. No. 12,584,903.
Application No. 18/170,644 is a continuation of application No. 16/551,505, filed on Aug. 26, 2019, now abandoned.
Application No. 16/551,505 is a continuation of application No. 15/529,814, filed on Nov. 23, 2015, now Pat. No. 10,393,731.

(30) Foreign Application Priority Data
Nov. 27, 2014 (EP) 14195277.0

(51) Int. Cl. G01N 33/50

(52) U.S. Cl. CPC G01N 33/5014; G01N 33/5064

(57) ABSTRACT

The present invention relates to cell-based methods for determining the biological activity of defibrotide. In particular, the invention provides a method for assessing the potency of defibrotide by assessing the viability of mammalian cells in the presence of at least one cytotoxic agent and one or more concentrations of defibrotide. Such methods are particularly useful for standardizing pharmaceutical compositions comprising defibrotide.

### PRIORITY

This application is a continuation application of U.S. application Ser. No. 18/170,644, filed Feb. 17, 2023, which is a continuation application of U.S. application Ser. No. 16/551,505, filed Aug. 26, 2019, which is a continuation application of U.S. application Ser. No. 15/529,814, filed May 25, 2017, which is a 371 National Stage Entry of International Application PCT/EP2015/077355, filed Nov. 23, 2015, which claims priority to EP 14195277.0 filed Nov. 27, 2014, the contents of each of which are herein incorporated by reference in their entireties.

### BACKGROUND OF THE INVENTION

Medicinal substances should be produced at a constant specific activity level so that they can be delivered safely. For example, assays for biological molecules such as heparin have variability from batch to batch in terms of chain length, molecular weight, composition, degree of sulphation, etc. Other substances that are extracted from natural substances also need to be standardized. See for example, U.S. Pat. No. 7,575,886. One such substance is defibrotide. Defibrotide is a heterogeneous mixture of single-stranded polynucleotides of varying lengths that is extracted from mammalian organs.

There are assays available to evaluate the biological activity of defibrotide, including the fibrin plate test and the thromboelastographic recording of the euglobulin lysis time (Prino G. et al., Indagini preliminari sull'attivitfibrinolitica, nell'animale e nell'uomo, di una nuova sostanza presente in diversi organi animali, Simposio Intemazionale: La ricerca scientifica nell'industria farmaceutica in Italia, Rome, 2-4 Oct. 1975-II Farmaco, Ed. Prat.) (1969),24,552-561), the plasmin method (U.S. Pat. No. 7,338,777), and the euglobulin method (WO2013/190582). Although these methods are useful pharmaceutical manufacturing tools, all these methods, which are based on the pro-fibrinolytic properties of defibrotide, involve an assessment of defibrotide's activity on isolated proteins or enzymes.

Thus, there is a need in the art for novel methods to determine the biological activity of defibrotide in a cellular context that provides an accurate and reliable process for assessing the potency, e.g., by comparison with a reference defibrotide standard preparation, of new batches of defibrotide regardless of the manufacturing process used.

### SUMMARY OF THE INVENTION

The present invention is based, in part, on the discovery that defibrotide protects mammalian cells from cytotoxicity induced by particular cytotoxic agents in a dose-dependent manner. The inventors have taken advantage of this cell protection effect and developed a cell-based method for assessing the potency of defibrotide batches and defined a measurement unit to facilitate effective and safe administration. Such methods allow for, inter alia, quality control during the defibrotide manufacturing process, standardization of defibrotide batches produced by different methods or sources, and consistent dosing of patients with defibrotide.

### BRIEF DESCRIPTION OF THE DRAWINGS

FIG. 1. Viability of HMEC cells incubated with fludarabine in the presence or absence of varying concentrations of defibrotide as measured by MTT assay. HMEC-1 were incubated with fludarabine (F-Ara) at 10 μg/ml in the presence or absence of varying concentrations of defibrotide (DF) (1 μg/ml-100 μg/ml) for 72 hr and the viability of the cells was measured with the MTT assay. Student t-test: § p<0.01, F-ara 10 μg/ml vs. control (ctr); *p<0.05, cells treated with DF at 1 μg/ml vs. F-ara 10 μg/ml; **p<0.001, DF at 10 and 100 μg/ml vs. F-ara 10 μg/ml.

FIG. 2. Viability of SK-HEP-1 cells incubated with doxorubicin in the presence or absence of varying concentrations of defibrotide as measured by CCK-8 assay. SK-HEP-1 cells were incubated with doxorubicin (Dox) at 0.1 μg/ml in the presence or absence of varying concentrations of defibrotide (DF) (1 μg/ml-100 μg/ml) for 72 hr and the viability of the cells was measured with the CCK-8 assay. Student t-test: § p<0.01, Dox 0.1 μg/ml vs. control (ctr); *p<0.01, cells treated with DF at 50 or 100 μg/ml vs. Dox 0.1 μg/ml.

FIGS. 3A-3C. Viability of HMEC-1 cells incubated with fludarabine in the presence or absence of varying concentrations of defibrotide, AC or ACTG as measured by MTT assay. HMEC-1 cells were incubated with fludarabine (F-Ara) at 50 μg/ml in the presence or absence of varying concentrations of random synthetic Adenine-Cytosine (AC) oligonucleotides of about 16 kDa (1-500 μg/ml) (FIG. 3A), random synthetic Adenine-Cytosine-Guanine-Thymine (ACGT) oligonucleotides of about 17 kDa (12.5-50 μg/ml) (FIG. 3B), or defibrotide (5-100 μg/ml) (FIG. 3C) for 72 hr and the viability of the cells was measured with the MTT assay. Student t-test: *p<0.01, F-ara 50 μg/ml vs. control (Ctr), **p<0.01 F-ara 50 μg/ml vs. defibrotide. There was no significant protection by either of the synthetic oligonucleotides.

FIG. 4. Viability of SK-HEP-1 cells incubated with fludarabine in the presence or absence of varying concentrations of ACTG, tpA, or glutathione as measured by CCK-8 assay. SK-HEP-1 cells were incubated with fludarabine (F-Ara) at 10 μg/ml in the absence or presence, (A-G), of varying concentrations of random synthetic Adenine-Cytosine-Guanine-Thymine (ACGT) oligonucleotides of about 17 kDa (1.25-80 μg/ml), tPA (10-320 IU/ml), or glutathione (1.25-80 μg/ml) for 72 hr and the viability of the cells was measured with the CCK-8 assay. Student t-test: *p<0.01, F-Ara 10 μg/ml vs. control (Ctr). There was no significant protection from F-Ara-induced cytoxicity by ACGT, tPA, or glutathione.

FIGS. 5A-5B. Comparison of the dose response curves of standard defibrotide versus acid-stressed (FIG. 5A) and basic-stressed (FIG. 5B) defibrotide samples in the cell protection assay. The raw absorbance data was processed using the PLA2 statistical analysis program (4-parameter logistic function analysis). Absorbance of the cell viability indicator dye (CCK-8) is plotted on the Y-axis as “response.” Dose is plotted on the X-axis and is a 2-fold dilution series of defibrotide in the assay (1.25-80 μg/ml); STD represents the reference standard defibrotide. The lower traces in each panel, which correspond to the stressed samples, indicate a reduced potency. Using this statistical analysis program, both stressed samples failed to meet the statistical criteria of acceptance.

FIG. 6. Viability of SK-HEP-1 cells incubated with fludarabine in the presence or absence of varying concentrations of defibrotide as measured by CCK-8 assay. SK-HEP-1 cells were incubated with fludarabine (F-Ara) at 10 μg/ml in the absence or presence of varying concentrations of defibrotide (DF) (1 μg/ml-100 μg/ml) for 72 hr and the viability of the cells was measured with the CCK-8 assay. Student t-test: p<0.01, F-Ara 10 μg/ml vs. control (Ctr) and for cells treated with DF at >1 μg/ml vs. F-Ara 10 μg/ml.

FIG. 7. Assessment of the potency ratio between a standardized Reference defibrotide sample (standard) and a sample of defibrotide of unknown biological activity. SK-HEP-1 cells were exposed to 6 serial dilutions (1:2) of standard and sample defibrotide to give concentration of 80, 40, 20, 10, 5, 2.5 and 1.25 μg/ml in the presence of fludarabine (F-Ara) (10 μg/ml). Each concentration of the standard and the sample consisted of 4 replicates. After 72 hr incubation at 37 °C, the viability of the cells was measured with the CCK-8 assay. The absorbance measurements were submitted to statistical analysis for sample potency determination (4-parameter logistic analysis).

### DETAILED DESCRIPTION OF THE INVENTION

The present invention provides a reliable method for determining the biological activity of defibrotide based on the ability of defibrotide to protect living cells from the effects of certain cytotoxic agents. This cell protection effect is important for defibrotide's use as a medicinal product. This method allows for the standardization of the activity for defibrotide that is obtained by different methods or sources. The method also allows for the establishment and assignment of a unit of measurement to facilitate effective and safe administration of defibrotide.

In one embodiment of the invention, the method of assessing the potency of a sample batch of defibrotide comprises (i) growing mammalian cells in culture, (ii) incubating the cells with a solution containing at least one cytotoxic agent and at least one concentration of defibrotide from the sample batch, (iii) determining the viability of the cells after an incubation period, and (iv) calculating the potency of the sample batch of defibrotide based on the cell viability measurement. In some embodiments, the method further comprises comparing the cell viability for the sample batch of defibrotide to the cell viability for a reference batch of defibrotide, and calculating the potency of the sample batch of defibrotide based on the comparison.

Defibrotide (Merck Index, 1996, no. 2915; CAS number 83712-60-1) is a substance of natural origin. It is the sodium salt of low molecular weight polydeoxyribonucleotides which are obtained by extraction from animal organs. Defibrotide is known to have a molecular weight (MW) between 14 and 19 kDa, but specific measurement techniques show defibrotide to have an average molecular weight (MW) around 16.1 kDa±2.0 kDa if determined by SEC-HPLC technique; a MW around 17.6 kDa±1.0 kDa if determined by PAGE technique; and a MW around 16.7 kDa±1.6 kDa if determined by Multi-Angle Laser Light Scattering technique. “Analysis of Aggregates and Particles in Protein Pharmaceuticals” H. Mahler and W. Jiskoot (eds.), 2012 John Wiley & Sons, Inc.

Defibrotide has numerous therapeutic applications, including use as an anti-thrombotic agent (U.S. Pat. No. 3,829,567), treatment of peripheral arteriopathies, treatment of acute renal insufficiency (U.S. Pat. No. 4,694,134), and treatment of acute myocardial ischaemia (U.S. Pat. No. 4,693,995). More recently, defibrotide has been used for the treatment and prevention of sinusoidal obstruction syndrome/venous occlusive disease (EU clinical trial EudraCT:2004-000592-33, US clinical trial 2005-01 (ClinicalTrials.gov identifier: NCT00358501). Other uses of defibrotide are described in the following patents and patent applications, each of which is hereby incorporated by reference in its entirety: U.S. Pat. Nos. 3,770,720; 3,829,567; 3,899,481; 4,693,134; 4,693,995; 4,938,873; 4,985,552; 5,081,109; 5,116,617; 5,223,609; 5,646,127; 5,646,268; 5,977,083; 6,046,172; 6,699,985; 6,767,554; 7,338,777; 8,551,967; 8,771,663, US Patent Publication Nos. 20080194506; 20090131362; 20110092576; 20130231470; 20140005256, U.S. patent application Ser. No. 14/323,918; and WO 2013/190582.

The methods described herein can be used to assess the potency of defibrotide batches manufactured by different methods or extracted from different animal organs. For instance, in some embodiments, the defibrotide sample batch is extracted from bovine tissue, such as bovine lung, intestine, or mucous membranes. In other embodiments, the defibrotide sample batch is extracted from porcine tissue, such as porcine lung, intestine, or mucous membranes. Defibrotide sample batches may also be extracted from other organs from other animal species, including sheep and horses.

In certain embodiments, the defibrotide sample batches to be evaluated for potency by the methods described herein are manufactured by a process such as that described in U.S. Pat. Nos. 4,985,552 and 5,223,609, both of which are hereby incorporated by reference in their entireties. In particular, the defibrotide obtained with this process is a polydeoxyribonucleotide corresponding to the following formula of random sequence:

P₁₋₅,(dAp)₁₂₋₂₄,(dGp)₁₀₋₂₀,(dTp)₁₃₋₂₆,(dCp)₁₀₋₂₀
wherein:
P=phosphoric radical
dAp=deoxyadenylic monomer
dGp=deoxyguanylic monomer
dTp=deoxythymidylic monomer
dCp=deoxycytidylic monomer.

The defibrotide sample batches may have one or more or all of the following chemico-physical properties: electrophoresis=homogeneous anodic mobility; extinction coefficient, E_{1cm}^{1%} at 260±1 nm=220±10; extinction ratio, E₂₃₀/E₂₆₀=0.45±0.04; coefficient of molar extinction (referred to phosphorus), ε(P)=7750±500; rotary power [α]D^{20°}=53°±6; reversible hyperchromicity, indicated as % in native DNA, h=15±5; and a purine:pyrimidine ratio of 0.95±0.5.

In certain embodiments, the defibrotide sample batches to be evaluated for potency by the methods of the invention may have been subjected to a physiochemical stress or suspected of being exposed to a physiochemical stress, such as high temperature, extreme pH, hydrogen peroxide, etc. Thus, the methods of the invention can also be used to assess the potency of defibrotide batches or compositions comprising defibrotide that have been stored at sub-optimum conditions or for extended periods of time. In certain embodiments, the methods can be used to monitor the stability of defibrotide batches or compositions comprising defibrotide, for example, to predict shelf-life.

The methods of the invention comprise growing mammalian cells in culture. In certain embodiments, the mammalian cells are human cells. In some embodiments, the human cells are human epithelial cells. In other embodiments, the human cells are human endothelial cells. In one particular embodiment, the human endothelial cells are human liver sinusoidal endothelial cells, such as SK-HEP-1 cells. In another particular embodiment, the human endothelial cells are human microvascular endothelial cells, such as HMEC-1 cells. In another particular embodiment, the epithelial cells are keratinocytes (e.g. HaCaT cells) or alveolar epithelial cells (e.g. A549 cells). Mammalian cells can be obtained from recognized depositories, such as the American Type Culture Collection (ATCC) as well as other sources.

Suitable growth media for growing mammalian cells in culture are well known in the art and are disclosed for instance in “Culture of Animal Cells: A Manual of Basic Technique and Specialized Applications” R. I. Freshney, 2010, Wiley-Blackwell. The optimal medium for each type of cells can be obtained from specialised suppliers of the cells (e.g.: ATCC-LGC, MI, Italy; CDC, Atlanta, GA, USA). In certain embodiments, the mammalian cells are grown in Eagle's Minimum Essential Medium (EMEM) supplemented with 10% (v/v) fetal bovine serum (FBS), 100 units/mL Penicillin, 100 g/mL Streptomycin, and 2.5 g/mL Amphotericin B. In other embodiments, the mammalian cells are grown in RPMI 1640 medium supplemented with 10% (v/v) fetal bovine serum (FBS), 100 units/mL Penicillin, 100 g/mL Streptomycin, and 2.5 g/mL Amphotericin B. In certain embodiments, the growth media may contain L-glutamine (e.g. 2 mM), hydrocortisone (e.g. 10 μg/ml), and epidermal growth factor (e.g. 10 μg/ml).

The density of the mammalian cells may be from about 5×10⁴ cells/ml to about 5×10⁵ cells/ml, from about 2.5×10⁴ cells/ml to about 2.5×10⁵ cells/ml, or from about 5×10⁴ cells/ml to about 2×10⁵ cells/ml. In order to obtain optimal assay response, the cell density may, in certain embodiments, be optimised taking into account the nature of the cytotoxic agent and the type of cell used for the assay. For example, in embodiments in which human endothelial cells are used for the assay, particularly suitable cell densities range from about 2.5×10⁴ cells/ml to about 2×10⁵ cells/ml, preferably about 5×10⁴ cells/ml. These cell densities are particularly suitable for assays in which doxorubicin or fludarabine is the cytotoxic agent.

In another aspect of the invention, the methods comprise incubating the mammalian cells in culture with a solution comprising a cytotoxic agent and at least one concentration of defibrotide from the sample batch under evaluation. As used herein, the term “cytotoxic agent” refers to a compound that has a toxic effect on a cell, such as inducing cell necrosis, inhibiting cell growth or cell division, or inducing cell apoptosis. The cytotoxicity of compounds can result from various properties, including, but not limited to, anti-metabolite properties, alkylating properties, nucleic acid intercalating properties, or apoptotic properties.

An anti-metabolite property is the ability of the compound, or its metabolites, to interfere with the proper synthesis of biomolecules, such as DNA and RNA. Examples of compounds having an anti-metabolite property include nucleobase analogs (e.g. purine and pyrmidine analogs), nucleoside and nucleotide analogs, and antifolate compounds. Exemplary nucleobase and nucleoside analogs that have cytotoxic effects include, but are not limited to, azathioprine, thiopurines (e.g. thioguanine, mercaptopurine), fludarabine, pentostatin, 5-fluorouracil, 6-azauracil, clofarabine, nelarabine, cladribine, cytarabine, floxuridine, capecitabine, gemcitabine, azacitidine, and decitabine. Examples of antifolates include methotrexate, aminopterin, pemetrexed, pralatrexate, and raltitrexed.

An alkylating property is the ability of the compound, or its metabolites, to transfer alkyl groups to biomolecules or form covalent bonds with reactive groups within biomolecules (e.g. amino, carboxyl, sulfhydryl, and phosphate groups), which can inactivate or interfere with their biological function. Many alkylating agents can cross-link DNA strands impairing DNA replication, which can lead to the induction of apoptosis. Examples of alkylating agents include nitrogen mustards (e.g. mechlorethamine, cyclophosphamide, melphalan, chlorambucil, ifosfamide and busulfan), nitrosoureas (e.g. N-Nitroso-N-methylurea, carmustine, lomustine, and semustine, fotemustine and streptozotocin), tetrazines (e.g. dacarbazine, mitozolomide and temozolomide), aziridines (e.g. thiotepa, mytomycin and diaziquone), and cisplatins (e.g. cisplatin, carboplatin and oxaliplatin).

A nucleic acid intercalating property is the ability of the compound, or its metabolites, to insert into the DNA double helix, which can cause mutations, or intercalate within regions of helical structures of RNA. Examples of intercalating agents include ethidium bromide, mitomycin, actinomycin, plicamycin, anthracyclines (e.g. doxorubicin, daunorubicin, epirubicin, idarubicin, valrubicin, and mitoxantrone), thalidomide and bleomicins.

An apoptotic property is the ability of the compound, or its metabolites, to induce programmed cell death. One particular class of compounds that can induce apoptosis is anti-microtubule agents, which interfere with mitosis and result in cell cycle arrest, thereby inducing apoptosis. Anti-microtubule agents include vinca alkaloids, such as vincristine, vinblastine, vinorelbine, vindesine, and vinflunine, and taxanes, such as paclitaxel and docetaxel.

Topoisomerase inhibitors also are cytotoxic by virtue of their ability to prevent DNA replication and transcription and/or by causing DNA strand breaks, thereby inducing apoptosis. Topoisomerase inhibitors include, but are not limited to, irinotecan, topotecan, etoposide, doxorubicin, mitoxantrone and teniposide.

The cytotoxic agent used in the methods of the invention is generally a synthetic, semi-synthetic, or natural chemical compound. The compound may have one or more of the properties described above. The cytotoxic agent can be any of the compounds described herein or a metabolite thereof. In some embodiments, the cytotoxic agent may be selected from fludarabine, cytarabine, 5-fluorouracil, methotrexate, busulfan, melphalan, cisplatin, ethidium bromide, doxorubicin, anthracyclines, thalidomide, or combinations thereof. In certain embodiments, the cytotoxic agent used in the methods of the invention is fludarabine or its active metabolite, 9-beta-D-arabinofuranosyl-2-fluoroadenine (F-Ara-A). In other embodiments, the cytotoxic agent used in the methods of the invention is doxorubicin.

Alternative cytotoxic agents commonly known to the person skilled in the art are equally suitable for use in the methods of the present invention. For example, in some embodiments, the cytotoxic agents slow or arrest cell cycle progression, and/or induce apoptosis of cells. Such types of cytotoxic agents include Staurosporine, Bendamustine, Carmustine, Imatinib and salts thereof (marketed as Gleevec), Ara-C, Gemtuzumab (such as Gemtuzumab ozogamicin, marketed as Mylotarg), Azacitidine (marketed as Vidaza), Decitabine (marketed as Dacogen), Vorinostat (marketed as Zolinza), and Thapsigargin, H₂O₂, and Phorbol Myristate Acetate. See also the NIOSH list of Antineoplastic and Other Hazardous Drugs in Healthcare Settings 2012, HHS, Publication No. 2012-150 (www.cdc.gov/niosh/docs/2012-150/pdfs/2012-150.pdf).

The concentration of the cytotoxic agent used in the methods of the invention will vary depending on the particular cytotoxic agent and mammalian cell type being used. In embodiments in which fludarabine or F-Ara-A is the cytotoxic agent, the agent is present in the growth medium at a final concentration from about 10 μg/ml to about 50 μg/ml. In other embodiments in which doxorubicin is the cytotoxic agent, the agent is present in the growth medium at a final concentration from about 0.1 μg/ml to about 10 μg/ml.

The cytotoxic agents may be used singly or in combination of 2, 3, 4, 5, 6, or more agents. In certain embodiments, the potency of a single sample batch of defibrotide may be assessed by evaluating independently its cell protection effect for two different cytotoxic agents. By way of example, a first potency value of the defibrotide sample batch may be obtained by performing the method with a first cytotoxic agent (e.g. fludarabine) and a second potency value may be obtained by performing the method with a second cytotoxic agent (e.g. doxorubicin). An overall potency of the defibrotide sample batch may be determined by a mathematical comparison of the first and second potency values, for example by averaging the two value or calculating a ratio of the two values.

In certain embodiments, a particular set of culture conditions may be used to induce cytotoxicity of the mammalian cells rather than employing a specific cytotoxic agent or agents. For instance, the methods may comprise exposing mammalian cells to an apoptosis-inducing culture medium in the presence of at least one concentration of defibrotide, determining the viability of the cells after an incubation period, and calculating the potency of the defibrotide based on the cell viability measurement. An apoptosis-inducing culture medium can include medium having an acidic pH (e.g. pH of about 2 to about 6 or about 4.5 to about 6.5) or a basic pH (e.g. pH of about 7.5 to about 10 or about 8 to about 9.5). Apoptosis-inducing culture medium also includes medium that does not contain essential growth factors (e.g. fibroblast growth factor, epidermal growth factor, platelet-derived growth factor) as withdrawal of growth factors is recognized as an inducer of apoptosis. As used herein, “apoptosis-inducing medium” can also refer to medium at a particular temperature range (e.g. greater than 37 °C) or oxygen concentration range (less than 5% oxygen) that induces apoptosis. The apoptosis-inducing culture medium or conditions can be readily adjusted by a person of ordinary skill in the art for the particular mammalian cell type being employed in the methods. U.V. or other types of radiation can also be used to induce apoptosis. In some embodiments, the incubation solution comprises at least one concentration of defibrotide from the sample batch under evaluation in addition to the cytotoxic agent. The concentration of defibrotide from the sample batch (e.g. final concentration in cell-containing medium) can be in the range from about 1 μg/ml to about 1 mg/ml, from about 1 μg/ml to about 100 μg/ml, from about 1.25 μg/ml to about 80 μg/ml, or from about 5 μg/ml to about 50 μg/ml.

In certain embodiments, multiple concentrations of the defibrotide from the sample batch are tested. For instance, in one embodiment, at least two different concentrations of defibrotide from the sample batch are separately tested. In another embodiment, at least three different concentrations of defibrotide from the sample batch are separately tested. In a particular embodiment, at least four different concentrations of defibrotide from the sample batch are separately tested. The multiple concentrations of defibrotide from the sample batch are preferably within the ranges disclosed above. In some embodiments, the multiple concentrations of the defibrotide from the sample batch are prepared by successive 1:2 dilutions of a stock solution.

In some embodiments, the method further comprises testing a reference defibrotide batch simultaneously with the defibrotide sample batch. The reference defibrotide batch is typically tested at various known concentrations of defibrotide. Multiple concentrations of the reference defibrotide batch may, in some embodiments, be tested. As with the multiple concentrations of the defibrotide sample batch, the multiple concentrations of the defibrotide reference batch can be prepared by serial dilution of a stock solution in accordance with a predetermined dilution factor. The concentrations of the defibrotide from the reference batch are preferably in the same concentration range as the concentrations from the defibrotide from the sample batch. For example, the concentrations of defibrotide from the reference batch (e.g. final concentration in cell-containing medium) can be from about 1 μg/ml to about 1 mg/ml, from about 1 μg/ml to about 100 μg/ml, from about 1.25 μg/ml to about 80 μg/ml, or from about 5 μg/ml to about 50 μg/ml.

In some embodiments of the method, at least 4 concentrations of the defibrotide sample batch and the defibrotide reference batch are prepared with at least 3 replicates for each concentration of the sample batch and reference batch.

In certain embodiments, the methods comprise a positive control of cytotoxicity. For instance, the mammalian cells are incubated with the cytotoxic agent alone (i.e. without any defibrotide) under the same conditions.

In some embodiments, the methods comprise a negative control of cytotoxicity. For example, in one embodiment, the mammalian cells are incubated in a solution without any defibrotide or cytotoxic agent under the same conditions. Such solutions may contain the cell growth medium and optionally any vehicle or solvent.

In one particular embodiment, the incubation of the mammalian cells with the cytotoxic agent with and without defibrotide (reference and sample batches, positive and negative controls) is conducted in a multi-well microtiter plate (e.g. 96-well). The subsequent determination of cell viability may also be performed in the microtiter plate. In some related embodiments, the wells of the microtiter plate are coated with a cell attachment matrix, such as poly-D-lysine.

The incubation period to obtain an acceptable assay response can be optimised, in relation to the cytotoxic agent and type of cell used in the method. One of skill in the art can adjust these parameters based on the common general knowledge.

In certain embodiments of the methods, the cells may be incubated with the cytotoxic agent and defibrotide from the sample and/or reference batches for a period ranging from about 12 to about 120 hours, from about 24 to about 96 hours, from about 48 to about 72 hours, or from about 48 to about 96 hours. In one embodiment, the incubation period is at least about 24 hours. In another embodiment, the incubation period is at least about 48 hours. In still another embodiment, the incubation period is at least about 72 hours.

Suitable incubation conditions for specific mammalian cell types can be found in general laboratory manuals, such as “Culture of Animal Cells: A Manual of Basic Technique and Specialized Applications” R. I. Freshney, 2010, Wiley-Blackwell. The set points for temperature and % CO₂ during the incubation period are other variables that can be adjusted to optimize the assay response. According to one embodiment of the present invention, the mammalian cells are incubated at a temperature ranging from between about 35 °C to about 39 °C. In another embodiment, the mammalian cells are incubated at a temperature ranging from between about 36 °C and about 38 °C. According to a further aspect of the present invention, the mammalian cells are incubated at a CO₂ concentration ranging from about 0 to about 10% (v/v) to maintain an optimal pH of the medium for cell growth. In another embodiment, the CO₂ concentration may be from about 1 to about 5%.

In another aspect of the methods of the invention, the viability of the mammalian cells is determined following incubation with the cytotoxic agent and defibrotide from a sample batch. Multiple techniques are available to assess cellular viability (see, e.g., Assay Guidance Manual, NCBI, 2013, G. Sitta Sittampalam et al. Eds., available on the Internet at www.ncbi.nlm.nih.gov/books/NBK53196/; Stoddart MJ., Cell viability assays: introduction; Methods Mol Biol. 2011; 740:1-6 and Riss et al., ASSAY and Drug Development Technologies, Vol. 2(1): 51-62, 2004, both of which are hereby incorporated by reference in their entireties), and any specific techniques described herein are illustrative only. In some embodiments, cell viability is assessed by using commercially available kits, such as the Cell Counting Kit 8 (Dojindo Molecular Technology Inc.; Sigma-Aldrich) and those available from Life Technologies (see www.lifetechnologies.com/us/en/home/references/molecular-probes-the-handbook/assays-for-cell-viability-proliferation-and-function.html) and Thermo Scientific (see C:UsersphillipnoucAppDataRoamingiManageWorkRecentJAZZ-008_03US-_306882.2653 Jazz Pharmaceuticals_Inc.—CELLULAR-BASED METHOD FOR DETERMINING THE BIOLOGICAL ACTIVITY OF DEFIBROTIt www.piercenet.com/product/alamarblue-cell-viability-assay-reagent).

Some suitable methods for determining cell viability that can be used with the methods of the invention include methods of assessing membrane integrity, assays measuring reduction or oxidation, methods that measure cellular ATP content, mitochondrial activity assays, and caspase assays. Methods of assessing membrane integrity (e.g. cytolysis or membrane leakage assays) include vital dye exclusion methods, such as those utilizing trypan blue, propidium iodide, erythrosin B or 7-Aminoactinomycin D, lactose dehydrogenase assays, and assays for protease biomarkers. Such methods generally entail measuring the presence of intracellular enzymes in the extracellular milieu (e.g. lactose dehydrogenase) or the presence of membrane impermeable dyes intracellularly as indications of compromised cell membranes.

Redox-based assays are typically colorimetric or fluorimetric methods in which certain classes of compounds (dyes/stains) change color or fluorescence as a result of biochemical reactions carried out by living cells. One example of these types of assays include the MTT assay in which cellular oxidoreductase enzymes reduce the tetrazolium dye MTT 3-(4,5-dimethylthiazol-2-yl)-2,5-diphenyltetrazolium bromide to its insoluble formazan, which has a purple color. Other closely related tetrazolium dyes can be used in similar assays to measure cellular viability. Thus, in certain embodiments of the methods of the invention, cell viability is determined by performing a colorimetric assay based on the reduction of tetrazolium dyes. Suitable tetrazolium dyes include 3-(4,5-dimethylthiazol-2-yl)-2,5-diphenyltetrazolium bromide (MTT), 2,3-bis-(2-methoxy-4-nitro-5-sulfophenyl)-2H-tetrazolium-5-carboxanilide (XTT), 3-(4,5-dimethylthiazol-2-yl)-5-(3-carboxymethoxyphenyl)-2-(4-sulfophenyl)-2H-tetrazolium (MTS), and water soluble tetrazolium salts, such as WST-1 and WST-8 (2-(2-methoxy-4-nitrophenyl)-3-(4-nitrophenyl)-5-(2,4-disulfophenyl)-2H-tetrazolium). Such techniques are well known in the art and are described, for instance, in Mosmann, “Rapid colorimetric assay for cellular growth and survival: application to proliferation and cytotoxicity assays,” J Immunol Methods. 1983 Dec. 16; 65: 55-63, which is hereby incorporated by reference in its entirety. A similar redox-based assay for determining cell viability utilizes the fluorescent dye, resazurin (7-Hydroxy-3H-phenoxazin-3-one 10-oxide). Resazurin is reduced to highly red fluorescent resorufin in live cells and thus cell viability can be determined by measuring the increase in fluorescence in the presence of the dye.

Cell viability can also be assessed by measuring changes in intracellular processes, such as changes in intracellular free radicals (e.g. reactive oxygen species, nitric oxide), free ion concentration (e.g. Ca²⁺, Mg²⁺, Zn²⁺), and membrane potential. Fluorescence indicators to monitor and quantitate such changes are commercially available from various sources, such as the fluorescent-based reagents available from Life Technologies and Promega. One such assay involves the use of calcein AM, which is a cell permeable dye that is a substrate for cellular esterases. Enzymatic activity in live cells converts calcein AM to a fluorescent product thereby allowing the determination of the number of live cells by increases in fluorescence. Quantitation of adenosine triphosphate (ATP) content has also been used as a marker of cell viability. Cellular ATP content can be measured by the amount of light produced through reaction with the luciferase enzyme using, for example, a luminometer.

In some embodiments, cell viability may be measured manually, for example by counting living cells with the aid of suitable microscopes or by means of suitable equipment evaluating the absorbance/fluorescence change, selected from a spectrophotometer, a spectrofluorimeter, a flow cytometer or a combination thereof. Other techniques for assessing cell viability are known to those of skill in the art and may be adopted for use with the methods of the present invention,

Depending on the assay used to assess cell viability, the cell viability measurement may be a change in the absorbance or fluorescence of the cell-containing solution or medium, a percentage or number of living cells, or a percentage or number of dead cells. In some embodiments, a change in absorbance or fluorescence can be converted into a percentage or number of living cells or dead cells. For instance, in embodiments in which a change in color or fluorescence occurs as a result of a dye or stain permeating a compromised cell membrane (e.g. trypan blue, erythrosin B or propidium iodide), an increase in absorbance or fluorescence at a particular wavelength indicates an increase in the number of dead cells. In other embodiments in which a change in color or fluorescence occurs as a result of a cellular reaction (e.g. MTT assay, calcein AM assay), the number of live cells correlates with an increase in absorbance or fluorescence at a particular wavelength. Thus, in certain embodiments of the methods of the invention, cell viability is determined by measuring the absorbance or fluorescence of the solution containing the mammalian cells following incubation with the cytotoxic agent and defibrotide. In one embodiment, the cell viability (e.g. absorbance or fluorescence) for each concentration of defibrotide from the sample batch (e.g. absorbance/fluorescence of each well of the microtiter plate containing different concentrations of defibrotide from the sample batch) is measured and plotted against the corresponding concentration of defibrotide to create a dose-response curve. In some embodiments, the dose-response curve is a sigmoidal curve (i.e. “S-shaped”). The range of defibrotide concentrations that are tested may be expanded or additional number of concentrations added to obtain a sigmoidal dose-response curve.

The absorbance or fluorescence readings or other cell viability measurements (e.g. number or percentage of live cells; e.g. number or percentage of dead cells) for each of the samples (e.g. the positive and negative controls, defibrotide reference batch and defibrotide sample batch), known as raw data, can be processed and subject to further statistical analysis. Dedicated software can be employed for statistical analysis, such as that especially designed for bioassay evaluation like, for example, PLA 2 (Stegmann Systems GmbH, Germany) or, alternatively, a commercial off-the shelf spreadsheet customised for the statistical evaluation of biological assay data.

In certain embodiments, the methods of the invention comprise comparing the cell viability measured for samples containing the sample batch of defibrotide to the cell viability measured for a reference batch of defibrotide. In some embodiments, the cell viability measurements for the reference batch of defibrotide were obtained prior to the analysis of the sample batch of defibrotide. Such prior measurements for reference batches of defibrotide can be stored in a reference database or computer readable storage medium. In certain embodiments the cell viability measurements for the reference batch represent an average of cell viability measurements obtained from a population of defibrotide reference batches. Thus, potency of the defibrotide sample batch can be calculated based on the cell viability measurements for the sample batch by comparison to a standard calibration curve obtained from prior analysis of a defibrotide reference batch or population of defibrotide batches. In other embodiments, the cell viability measurements for the reference batch of defibrotide are acquired at the same time as the cell viability measurements for the sample batch of defibrotide. For example, a series of concentrations for the defibrotide sample batch are run in parallel with a series of concentrations for the defibrotide reference batch.

The reference batch of defibrotide is preferably a standardized defibrotide batch having a known biological activity (e.g. pro-fibrinolytic activity, cell protection activity). For instance, in one particular embodiment, the reference batch of defibrotide has a cell protection activity of between 630-905 Units/mg. The standardized defibrotide batch can have one or more of the following characteristics: an average molecular weight of between about 14 to about 19 kDa as measured by SEC-HPLC, an extinction coefficient (ε1%) of about 207-233, an extinction ratio (Emin/Emax) of about 0.41-0.49, a purine to pyrimidine ratio greater than about 0.80 (e.g. about 0.80 to about 1.50), coefficient of molar extinction (F(P), referred to phosphorus) of about 7200 to about 8400, rotary power ([α]D^{20°}) of about 450 to about 60°, and reversible hyperchromicity, indicated as % in native DNA (h) of about 8 to about 22. In some embodiments, the reference batch of defibrotide is a batch of defibrotide manufactured under GMP conditions for clinical use. In other embodiments, the reference batch of defibrotide is a commercial batch of defibrotide available from Gentium (Villa Guardia, Italy).

In some embodiments, cell viability measurements for multiple concentrations of defibrotide from the reference batch are acquired to create a calibration curve. In one embodiment, creation of the calibration curve comprises the acquisition of the absorbance data relating to the samples at known increasing concentrations of defibrotide from the reference batch and the statistical processing of those data to obtain the calibration curve, which represents the correlation between the increase in cell viability in the presence of a cytotoxic agent and the dose of defibrotide. In certain embodiments, the cell viability measured for the sample batch of defibrotide is compared to a calibration curve obtained from cell viability measurements with a reference batch of defibrotide to determine the potency of the sample batch of defibrotide.

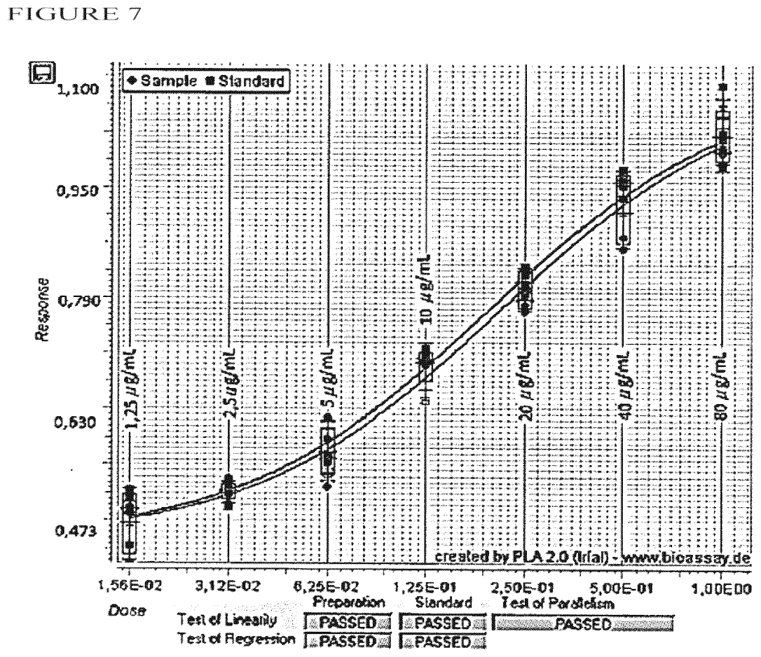

In some embodiments, a dose-response curve obtained from cell viability measurements from the defibrotide sample batch is compared to the calibration curve obtained from cell viability measurements with a reference batch of defibrotide. In such embodiments, the dose-response curve and the calibration curve may be sigmoidal curves (see FIG. 7, for example). The difference between the two curves is a function of the difference in biological activity between the defibrotide sample batch and the defibrotide reference batch. This difference is the potency of the sample batch of defibrotide compared to the reference batch. In one embodiment, a four-parameter logistic function model (4-PL, European Pharmacopoeia, section 5.3.2) is used to determine the difference between the dose-response curve for the defibrotide sample batch and the calibration curve for the defibrotide reference batch to calculate the potency of the sample batch of defibrotide. In another embodiment, a five-parameter logistic function model (5-PL, R. A. Dudley et al., “Guidelines for immunoassay data processing,” Clin. Chem., 1985, 31: 1264-1271) is used to determine the difference between the dose-response curve for the defibrotide sample batch and the calibration curve for the defibrotide reference batch to calculate the potency of the sample batch of defibrotide.

In some embodiments, the data obtained from the cell viability measurements of samples with the defibrotide sample batch can be evaluated for additional statistical parameters to ensure the data are valid. For example, the data can be required to satisfy certain statistical criteria, such as those mandated by regulatory agencies. Such tests may include tests for linearity, parallelism, and linear regression at the significance level of, for example 0.05, such that, F_{non-linearity}<F_{critical}, Fₙₒₙ₋ₚₐᵣₐₗₗₑₗᵢₛₘ<F_{critical}, and F_{Regression}>F_{critical}, respectively as detailed in for example, European Pharmacopoeia, section 5.3.2, 2014 and United States Pharmacopeia Chapter (1034) Analysis of Biological Assays, 2014.

The potency of the defibrotide sample batch calculated from statistical methods above can be expressed as a percent of the defibrotide reference batch, protection activity units per weight of defibrotide, or other units that may or may not be arbitrary. In some embodiments, a defibrotide protection unit is the concentration of defibrotide that mediates half-maximal cell-protection of SK-HEP-1 cells in the presence of 10 μg/ml fludarabine under the given assay conditions. In one embodiment, the potency of the defibrotide sample batch is expressed as a potency ratio relative to the potency of the reference batch of defibrotide. In certain embodiments, the potency ratio for the defibrotide sample batch is calculated using the following formula:

Potency Ratio=C_{ref}/Cₛₐₘₚ

Where, C is the concentrations of reference (ref) and sample (samp) defibrotide materials required to achieve the same effect.

The methods of assessing potency of a defibrotide sample batch as described herein can be used in the preparation of pharmaceutical compositions comprising defibrotide to adjust the quantity of defibrotide included in the compositions to ensure the compositions comprise accurate and consistent dosages. Thus, the methods of the invention can be used during the manufacturing process to assess the potency of different batches of defibrotide prepared at different locations, by different methods, or from different sources.

The methods of the invention can also be used to monitor the stability of defibrotide batches or pharmaceutical compositions comprising defibrotide over time. For instance, the potency of a batch or composition can be determined by the methods described herein periodically over time (e.g., monthly, biannually, annually) or following exposure to extreme conditions to monitor the activity of the defibrotide and identify batches or compositions that have deteriorated or degraded.

These and other aspects of the invention will be better illustrated in the following examples, which are not, however, to be regarded as limiting the invention.

All patent and non-patent documents referenced throughout this disclosure are incorporated by reference herein in their entirety for all purposes.

### EXAMPLES

### Materials and Methods

The following materials were used in the Examples given here below.

### Apparatus

A Victor 3 microplate reader equipped with different emission and absorption filters (Perkin Elmer, Milan, Italy), operated by Wallac software (Perkin Elmer, Milan, Italy).

Single and multi-channel pipettes with continuous volume adjustment equipped with sterile tips for molecular biology (Gilson, Milan, Italy.

Cell incubator with temperature and CO₂ control (Thermo Fischer Scientific, Milan, Italy.

Laminar flow hoods for tissue culture model HERAcell-150 (Thermo Fischer Scientific, Milan, Italy).

Analytical balance AX 26 DR (Mettler, Milan, Italy).

pH meter model 780 (Metrohom Italia, Milan, Italy).

Cell culture flasks, 25 and 75 cm², vent cap (Corning Incorporated, NY, USA).

Sterile, clear 96 well poly-D-lysine coated or uncoated (Sigma Aldrich, Milan, Italy).

Neubauer counting chamber and optical microscope (Carl Zeiss, Milan, Italy).

Vacuum medium filter sterilization unit (Sigma Aldrich, Milan, Italy?).

### Computer Programs

Microsoft Excel 2003. (Microsoft Corporation, Redmond, Wash., USA)
PLA 2 (Stegmann Systems GmbH, Germany) Cells
Human Microvascular Endothelial Cell line (HMEC-1, CDC, Atlanta, GA, USA)
Liver sinusoidal endothelial cell line (SK-HEP-1, ATCC, Manassas, VA, USA)

### Reagents and Chemicals

Defibrotide (Gentium, Italy)
9-beta-D-arabinofuranosyl-2-fluoroadenine, analytical grade (Sigma Aldrich, Milan, Italy), referred to as fludarabine or F-ara in the Figures and Examples below
Doxorubicin, analytical grade (Sigma Aldrich, Milan, Italy)
Amphotericin B (Sigma-Aldrich, Milan, Italy)
Dimethyl sulfoxide (DMSO) (Sigma-Aldrich, Milan, Italy)
Gelatin, 2% in water, tissue culture grade (Sigma-Aldrich, Milan, Italy)
Dulbecco's phosphate buffered saline (D-PBS) (Sigma-Aldrich, Milan, Italy)
Ethanol Absolute (Sigma-Aldrich, Milan, Italy)
Fetal Bovine Serum (FBS) (Sigma-Aldrich, Milan, Italy)
Penicillin-Streptomycin 100× (Sigma-Aldrich, Milan, Italy)
MTT (Sigma-Aldrich, Milan, Italy)
CCK-8 (Sigma Aldrich, Milan, Italy)
Trypan Blue (Sigma-Aldrich, Milan, Italy)
Eagle's Minimum Essential Medium (EMEM) ATCC Number: 30-2003 (ATCC Manassas, VA, USA)
Oligonucleotide (ACGT)ₙ of about 17 Kda (Sigma Genosys, Milan, Italy)
Oligonucleotide (AC)ₙ of about 17 KDa (Sigma Genosys, Milan, Italy)
Glutathione (Sigma-Aldrich, Milan, Italy)
Human tissue plasminogen activator (tPA) (Sigma-Aldrich, Milan, Italy)
Molecular biology grade water (Sigma-Aldrich, Milan, Italy)

### Preparation of Cell Growth Medium for SK-HEP-1

Cell growth medium for SK-HEP-1 was Eagle's Minimum Essential Medium (EMEM) supplemented with 10% (v/v) of foetal bovine serum (FBS), 1× Penicillin-Streptomycin and 1× Amphotericin B. From 500 ml of EMEM medium 65 ml was removed and 50 ml of FBS, 5 ml of a 100× concentrate of Penicillin-Streptomycin stock and 10 ml 50× concentrate of Amphotericin B stock was added. The medium was filter sterilised using a medium filter sterilisation unit.

### Preparation of Cell Growth Medium for HMEC-1

Cell growth medium for HMEC-1 was RPMI 1640 medium supplemented with 10% (v/v) FBS, 1× Penicillin-Streptomycin and 1× Amphotericin B. From 500 ml of RPMI 1640 medium 65 ml was removed and 50 ml of FBS, 5 ml of a 100× concentrate of Penicillin-Streptomycin stock and 10 ml 50× concentrate of Amphotericin B stock, 2 mM L-glutamine, 10 μg/ml hydrocortisone was added. The medium was filter sterilised using a medium filter sterilisation unit and sterile epidermal growth factor was added to a concentration of 10 μg/ml.

### SK-HEP-1 Cultivation and Preparation

The human liver sinusoidal endothelial cell line SK-HEP-1 was obtained from the American Type Culture Collection (ATCC) and cultured in complete EMEM medium in a humidified cell incubator containing 5% CO₂ at 37 °C using gelatin-coated tissue culture flasks. The cells were sub-cultured by trypsin mediated detachment every 2-3 days following the instructions provided by the ATCC. Cells were serially transferred into culture flasks when the culture was 80-90% confluent and used for the protection assay between passages +3 to +10. That is, 3 to 10 passages beyond the characterised passage number of the cells received from the ATCC.

A suspension of SK-HEP-1 for use in the cell protection assay was prepared and counted. Briefly the cells were washed with D-PBS, and detached using 1 ml of trypsin solution and resuspended in complete medium to a cell concentration of 10⁵, 2×10⁵ or 4×10⁵ cells/ml. Cells were counted using a Neubauer counting chamber in the presence of trypan blue to assess the percent viability of the cultures. The cell culture used in the cell protection assay had a viability of >90%.

### HMEC-1 Cultivation and Preparation

The human microvascular endothelial cell line (HMEC-1) was obtained from the Centers for Disease Control and Prevention (CDC) and cultured in complete RPMI 1640 medium in a humidified cell incubator containing 5% CO₂ at 37 °C. The cells were sub-cultured by trypsin mediated detachment every 2-3 days and serially transferred into culture flasks when the culture was 80-90% confluence and used for the protection assay between passages +3 to +10.

A suspension of HMEC-1 for use in the cell protection assay was prepared and counted. Briefly the cells were washed with D-PBS, and detached using 1 ml of trypsin solution and resuspended in complete medium to a cell concentration of 10⁵, 2×10⁵ or 4×10⁵ cells/ml. Cells were counted using a Neubauer counting chamber in the presence of trypan blue to assess the percent viability of the cultures. The cell culture used in the cell protection assay had a viability of >90%.

Preparation of Stock solutions

### 1. Fludarabine

A 10 mg vial of Fludarabine was dissolved in 1 ml of DMSO to give a solution of 10 mg/ml and stored at 4 °C. The stock solution was diluted 1:1 with complete growth medium to give the working stock solution of 5 mg/ml.

### 2. Defibrotide

Defibrotide stock solution was prepared on the day of use. Approximately 100 mg of defibrotide drug substance was accurately weighed into a 50 ml sterile tube and dissolved in 20 ml of D-PBS to give a solution of 5 mg/ml. This solution was diluted 1:10 with complete growth medium to give the working stock solution of 0.5 mg/ml used to produce the concentration dilution series.

3. Tissue Plasminogen Activator (tPA)

The content of two 10 μg vials of t-PA (about 400,000 IU/mg per vial) were dissolved in 2 ml of D-PBS to give a solution of 4000 IU/ml and stored at −80 °C.

### 4. ACGT Oligonucleotide,

A 1 mg vial of ACGT oligonucleotide was dissolved in 2 ml of D-PBS to give a solution of 0.5 mg/ml and stored at 4 °C.

### 5. Glutathione

100 mg of glutathione were dissolved in 20 ml of PBS? and diluted 1:10 with complete growth medium to a final concentration of 0.5 mg/ml.

### 6. Doxorubicin

A 10 mg vial of doxorubicin was dissolved in 10 ml DMSO and stored at −80 °C. A working stock was prepared by dilution in complete medium to 200 μg/ml.

### Plate Depositions

Fifty μl of the cell suspensions, or medium alone for blanks, prepared at the concentrations described above was placed in wells of a poly-D-lysine coated 96-well microtiter plate. The plates were placed in the cell incubator for 3 hr after which 50 μl of the challenge solution was added to cell-containing wells. Three or four replicate wells were used for each experimental condition. For example, the preparation of solutions containing fludarabine in the absence or presence of defibrotide is given in Table 1. Following addition of the solution to the wells, the plates were returned to the incubator and cell viability was assessed after 24, 48 or 72 hr. For a background measurement, 100 μl of complete medium alone was included in 3 to 4 replicate wells.

TABLE 1
Preparation of fludarabine and defibrotide solutions

| | Defibrotide | | Fludarabine | Defibrotide |
|---|---|---|---|---|
| Sample | (*) | Medium | Stock | Stock |
| type | (μg/ml) | (μl) | (μl) | (μl) |
| Negative | 0 | 3486 | 14 | 0 |
| Control | | | | |
| Defibrotide | 1.25 | 3468.5 | 14 | 17.5 |
| Sample 1:1 | | | | |
| Defibrotide | 2.5 | 3451 | 14 | 35 |
| Sample 1:2 | | | | |
| Defibrotide | 5 | 3416 | 14 | 70 |
| Sample 1:4 | | | | |
| Defibrotide | 10 | 3346 | 14 | 140 |
| Sample 1:8 | | | | |
| Defibrotide | 20 | 3206 | 14 | 280 |
| Sample | | | | |
| 1:16 | | | | |
| Defibrotide | 40 | 2926 | 14 | 560 |
| Sample | | | | |
| 1:32 | | | | |
| Defibrotide | | | | |
| Sample | | | | |
| 1:64 | 80 | 2366 | 14 | 1120 |
| Blank | 0 | 3500 | 0 | 0 |

(*) final concentration in each well following addition of 50 μL of cell suspension and 50 μL of the indicated solution

### Cell Viability Using MTT Assay

After the specified period of incubation, cell viability in each well was measured using the MTT assay. The MTT assay is based on the cleavage of tetrazolium salts by mitochondrial dehydrogenase in viable cells leading to the production of an insoluble formazan dye. MTT dye, 10 μl of a 2 mg/ml solution in D-PBS, was added to each well and then the plates were incubated for 3 hours. Plates were then centrifuged and each well aspirated. The dye was solubilised with 200 μl of a mixture DMSO/Ethanol (1:1) and absorbance in the wells was read at 570-590 nm on a microplate reader. A blank well containing only media and cytotoxic drug (fludarabine or doxorubicin) was also run as a control in all experiments.

### Cell Viability Using CCK-8 Assay

After the specified period of incubation, cell viability in each well was measured using the CCK-8 cell counting kit (Sigma Aldrich, Milan, Italy) following the manufacturer's instructions. The assay is based on the reduction by dehydrogenase activities of viable cells of the water soluble tetrazolium salt WST-8 (2-(2-methoxy-4-nitrophenyl)-3-(4-nitrophenyl)-5-(2,4-disulfophenyl)-2H-tetrazolium). The reduced formazan dye is soluble in tissue culture media. The amount of the formazan is directly proportional to the number of viable cells. The detection sensitivity of CCK-8 is higher than the other tetrazolium salts such as MTT. Unlike MTT, no solubilisation step is required and thus the assay can be measured continuously.

Briefly, after the specified incubation time, 10 μl of the supplied reagent was added to each well of the microtitre plate and the plate returned to the incubator. After 3 hours, the absorbance was measured at 450 nm with a background correction at 590 nm. Absorbance of the medium blank was subtracted from the test samples.

### Example 1

The present example shows the magnitude of the protection effect of defibrotide against fludarabine-induced cytotoxicity of HMEC-1 cells at physiologically relevant concentrations of fludarabine and defibrotide.

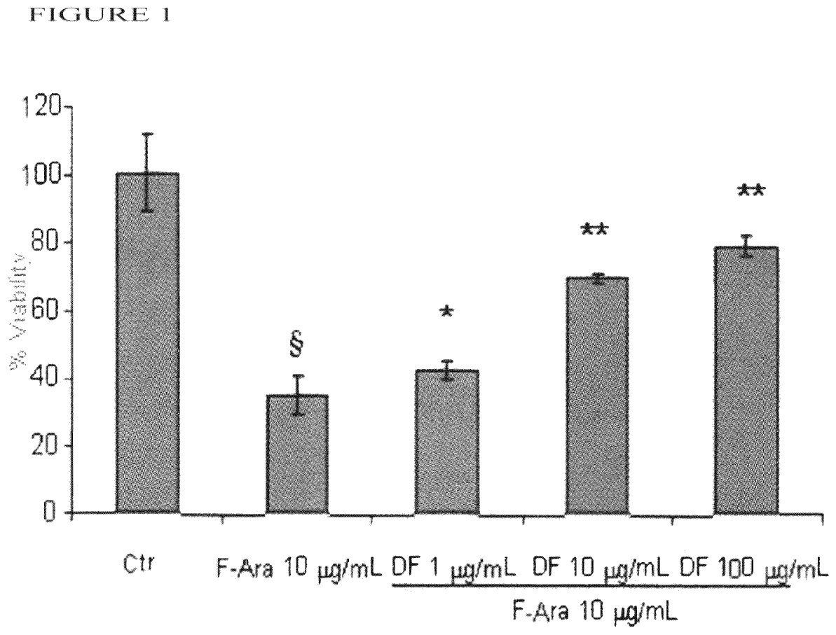

HMEC-1 cells were cultivated according to the above mentioned procedure. A cell density of 500,000 cells/ml was used for the assay. The cytotoxic agent was fludarabine at a concentration of 10 μg/ml. Defibrotide was added to the microplate well at a concentration of 100, 10 or 1 μg/ml. Four replicates of each condition were performed. The viability of the HMEC cells was assessed after 72 hours with the MTT assay described above. Defibrotide protected the cells from fludarabine-induced cytotoxicity in a dose-dependent manner with a protection effect of more than 50% observed with 100 μg/ml of defibrotide (FIG. 1).

### Example 2

The present example shows the magnitude of the protection effect of defibrotide against doxorubicin-induced cytotoxicity of SK-HEP-1 cells at physiologically relevant concentrations of doxorubicin and defibrotide.

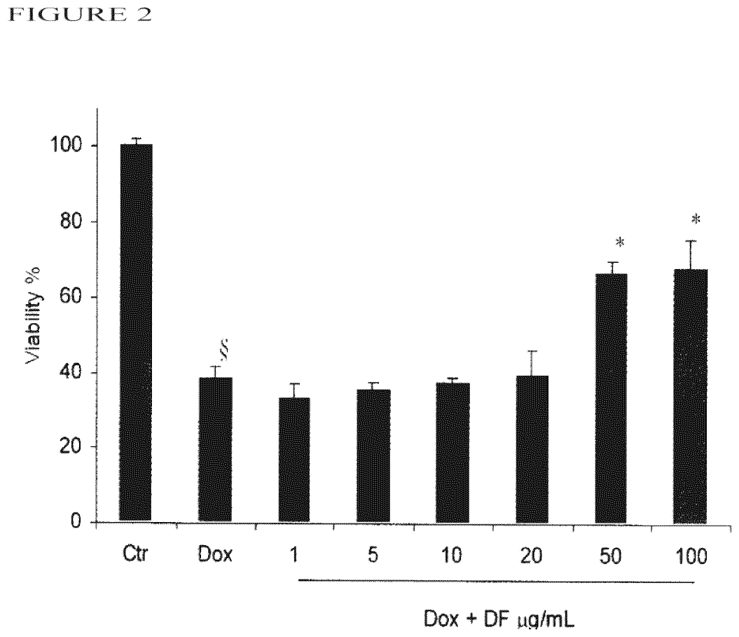

SK-HEP-1 cells were grown according to the above mentioned procedure. A cell density of about 50,000 cells/ml was used for the experiment. The cytotoxic agent was doxorubicin at a concentration of 0.1 μg/ml. Defibrotide was added to the microplate well at a concentration of 100, 50, 20, 10, 5 or 1 μg/ml. Three replicates of each condition were performed. The viability was assessed after 72 hours with the CCK-8 assay kit as described above. At concentrations of 50 μg/ml or greater, defibrotide significantly protected SK-HEP-1 cells from doxorubicin-induced cytotoxicity. (FIG. 2).

### Example 3

The present example compares the protective effective against fludarabine-induced cytotoxicity of defibrotide and synthetic oligonucleotides having similar average length and base composition to defibrotide.

HMEC-1 cells were cultivated according to the above mentioned procedure. A cell density of about 500,000 cells/ml was used for the experiment. The cytotoxic agent was fludarabine at a concentration of 50 μg/ml. Synthetic oligonucleotides (Adenine-Cytosine (AC) of about 16 kDa or Adenine-Cytosine-Guanine-Thymine (ACGT) of about 17 kDa) or defibrotide were added to each well at varying concentrations. Specifically, the AC oligonucleotides were added to each well at a concentration of 1, 10, 100 or 500, whereas the ACGT oligonucleotides were added to each well at a concentration of 12.5, 25 or 50 μg/ml. Defibrotide was added to each well at a concentration of 5, 25, 50, or 100 μg/ml. Each treatment condition was performed in triplicate. The viability was assessed with the MTT assay after 24, 48 and 72 hours.

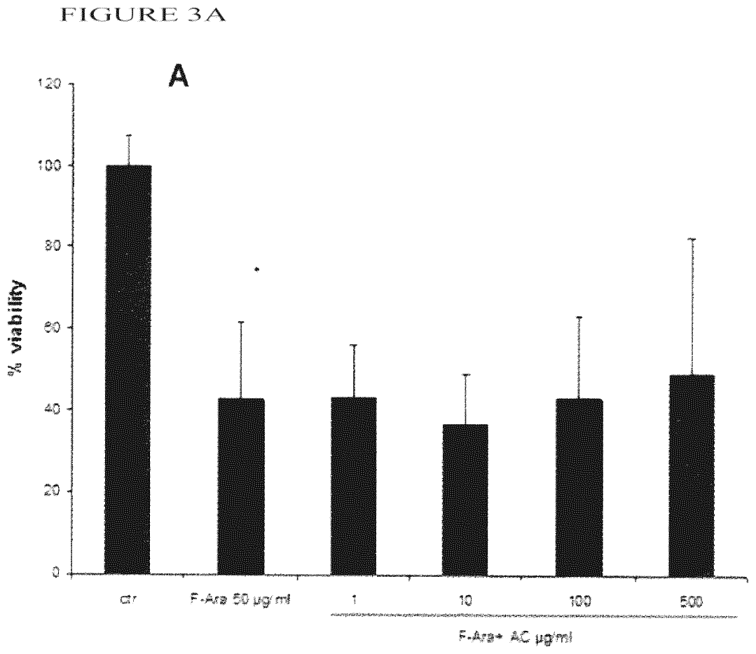

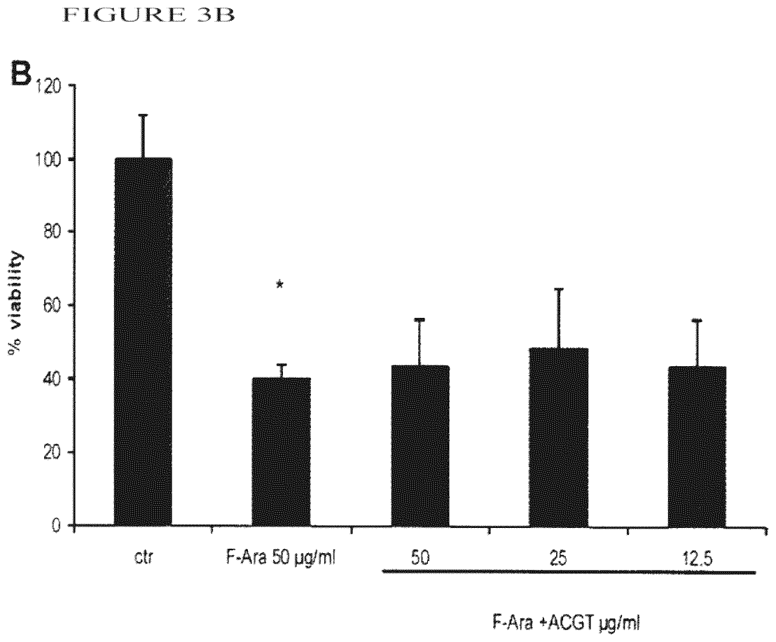

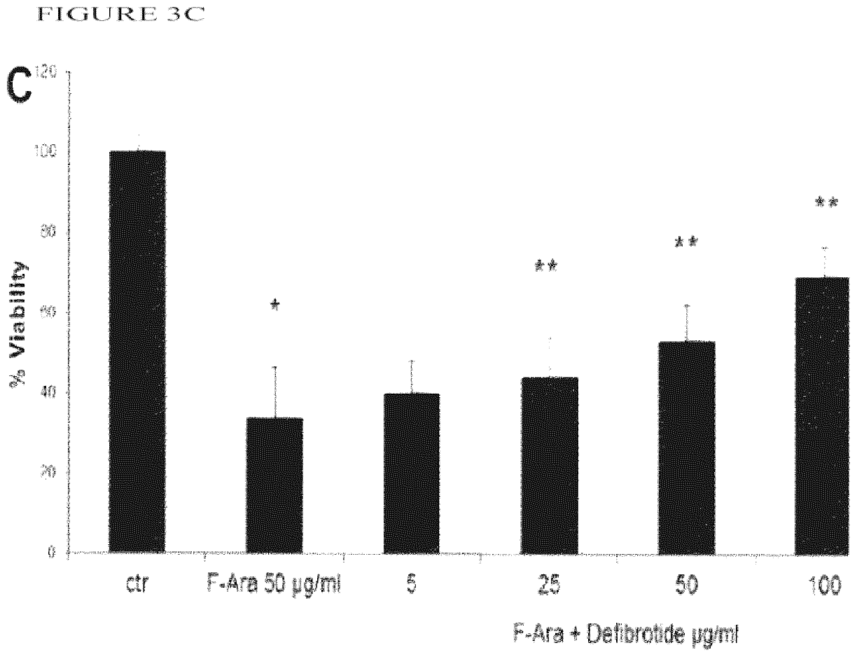

As shown in FIGS. 3A and 3B, neither the AC oligonucleotides nor the ACGT oligonucleotides had any protective effect against fludarabine-induced cytotoxicity of HMEC-1 cells after 72 hours of incubation. In contrast, defibrotide exhibited a dose-dependent protection of the cells from fludarabine-induced cytotoxicity (FIG. 3C).

### Example 4

The experiments described in this example tested the ability of a synthetic ACGT oligonucleotide, tPA, and glutathione to protect SK-HEP-1 cells from fludarabine-induced toxicity.

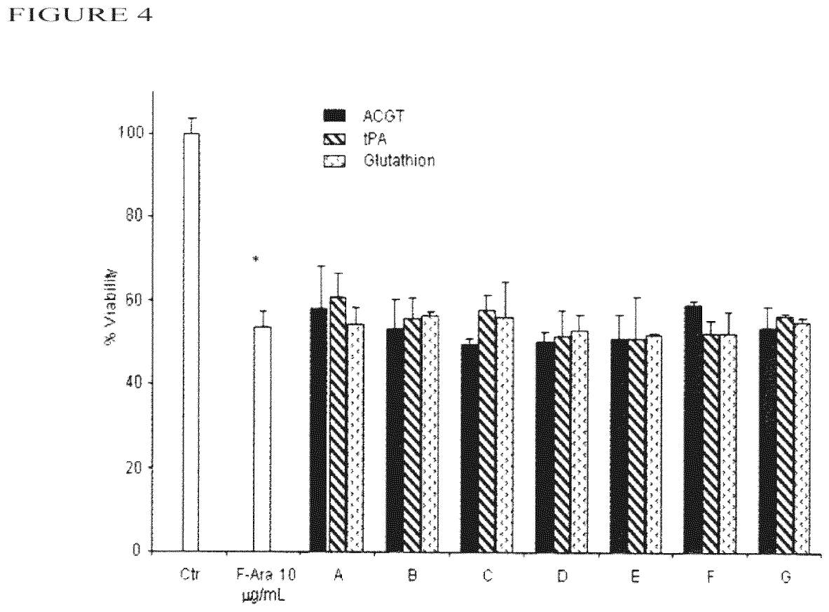

SK-HEP-1 cells were grown and expanded according to the above mentioned procedure. A cell density of about 50,000 cells/ml was used for the experiment. The cytotoxic agent was fludarabine at a concentration of 10 μg/ml. A random synthetic oligonucleotide (ACGT), tPA, or glutathione was added to each well at varying concentrations. Specifically, the ACGT oligonucleotide or glutathione was added to each well at a concentration of 1.25, 2.5, 5, 10, 20, 40, or 80 μg/ml, whereas tPA was added at a concentration of 10, 20, 40, 80, 160, or 320 IU/ml. Each treatment condition was performed in triplicate. The viability was assessed with the CCK-8 assay kit after 72 hours incubation. No protection of the SK-HEP-1 cells from fludarabine-induced cytotoxicity was observed with any of the three compounds (FIG. 4).

### Example 5

The present example evaluates the protective effective against fludarabine-induced cytotoxicity of defibrotide which has been modified as a result of physicochemical stress.

Defibrotide samples were stressed by submitting a standard sample of defibrotide to either 1) an Acidic Stress or 2) a Basic Stress. The Acidic Stress entailed incubating the standard defibrotide sample in a phosphate buffer having a pH of about 3 at about 80 °C for 18 hours. The Basic stress involved incubating the standard defibrotide sample in a phosphate buffer having a pH of about 12 at about 80 °C for 18 hours. After the incubation period, the solutions were brought to neutrality with phosphoric acid or sodium hydroxide.

SK-HEP-1 cells were grown according to the above mentioned procedure. A cell density of about 50,000 cells/ml was used for the experiment. The cytotoxic agent was fludarabine at a concentration of 10 μg/ml. Standard defibrotide (unmodified), defibrotide submitted to acid stress, or defibrotide submitted to basic stress were added to each well at a concentration of 80, 40, 20, 10, 5, 2.5, or 1.25 μg/ml. Each treatment condition was performed in triplicate.

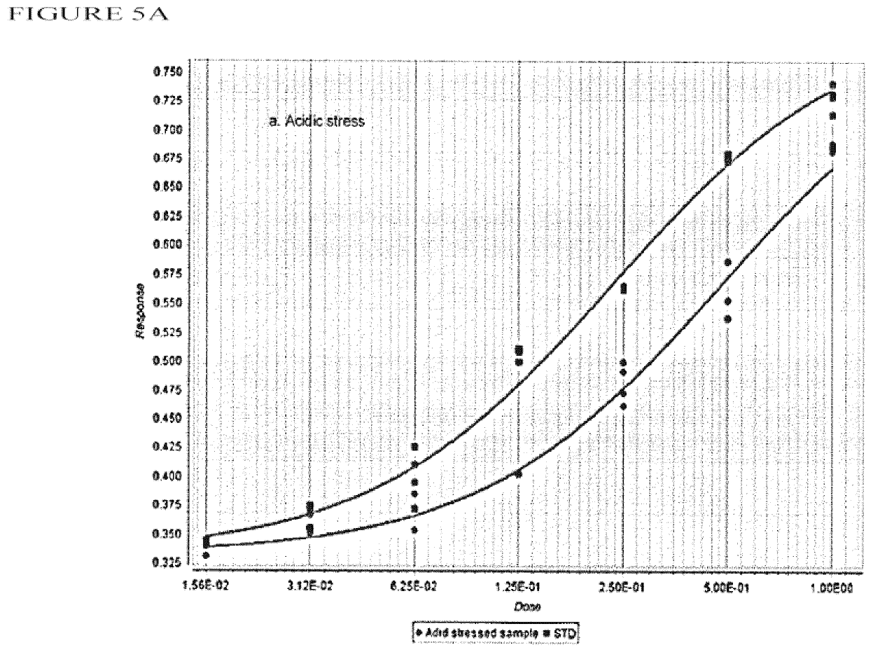

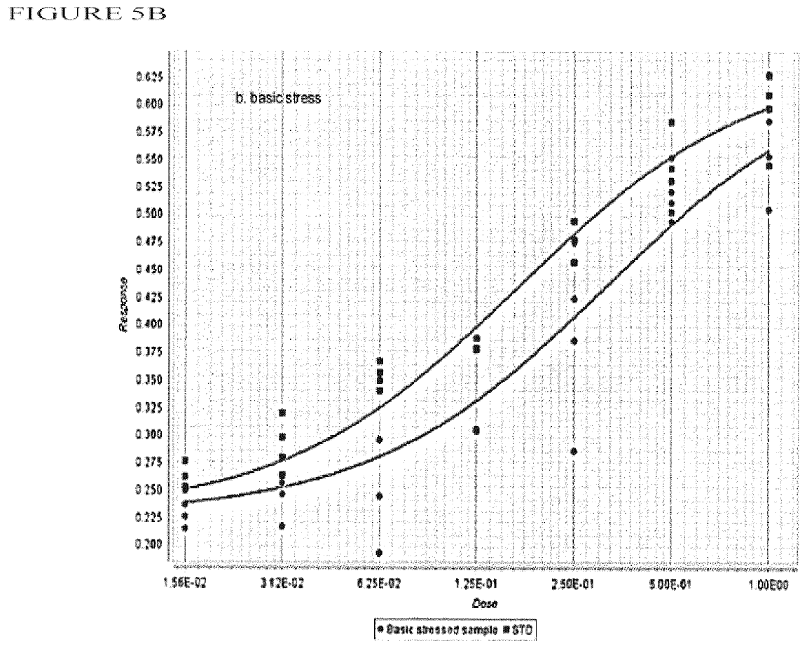

The viability of the cells was assessed with the CCK-8 assay kit after 72 hours incubation. Dose-response curves were constructed for unmodified defibrotide, acid-stressed defibrotide, and basic-stressed defibrotide. A comparison of the dose-response curves is shown in FIG. 5. Both the acid-stressed and basic-stressed defibrotide samples were less potent than unmodified defibrotide in protecting SK-HEP-1 cells from fludarabine-induced cytotoxicity (FIG. 5).

### Example 6

The present example shows the magnitude of the protection effect of defibrotide against fludarabine-induced cytotoxicity of SK-HEP-1 cells at physiologically relevant concentrations of defibrotide and fludarabine.

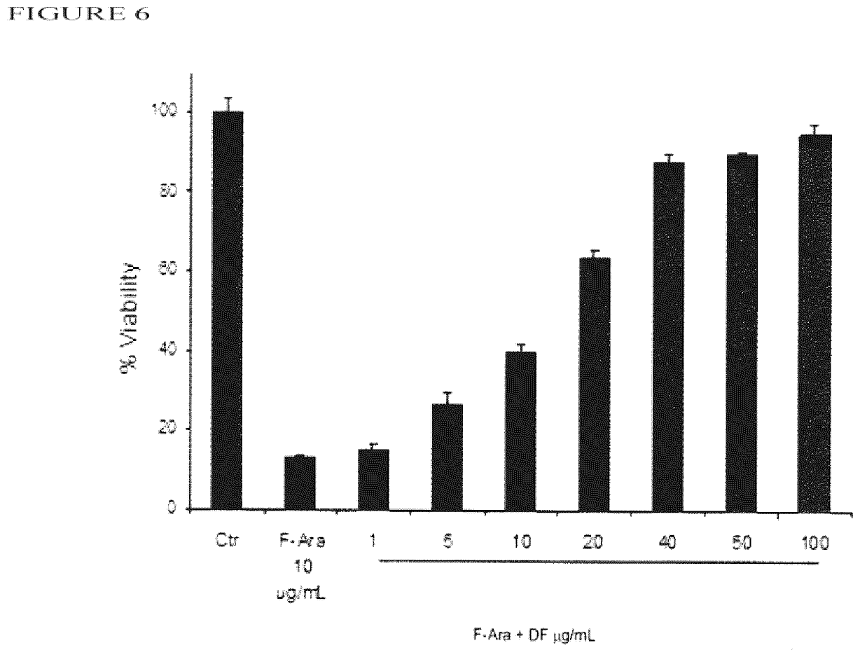

SK-HEP-1 cells were grown and expanded according to the above mentioned procedure. The cells at a cell density of about 50,000 cells/ml were plated in a poly-D-lysine coated 96-well microplate. The cytotoxic agent was fludarabine at a concentration of 10 μg/ml. Defibrotide was added to each well at a concentration of 100, 50, 40, 20, 10, 5 or 1 μg/ml. Each treatment condition was run in 4 replicate wells. The viability was assessed after 72 hours of incubation with the CCK-8 assay kit. Defibrotide produced a dose-dependent cell protection effect from fludarabine-induced cytotoxicity with greater than 80% of the cells surviving with concentrations of defibrotide of 40 μg/ml or greater (FIG. 6).

### Example 7

The present example shows the application of the cell-based protection assay for the assessment of the potency of a defibrotide sample of unknown biological activity.

SK-HEP-1 cells were grown and expanded according to the above mentioned procedure. The cells were plated at a cell density of about 50,000 cells/ml in poly-D-lysine-coated microplates. The cytotoxic agent was fludarabine at a concentration of 10 μg/ml. The defibrotide reference standard and the defibrotide test sample were added to separate wells at a concentration of 80, 40, 20, 10, 5, 2.5, or 1.25 μg/ml. Four replicate wells were run for each treatment condition. The viability of the cells was assessed with the CCK-8 assay kit after 72 hours of incubation.

The absorbances measured for the defibrotide reference standard samples and the defibrotide test samples were submitted to a 4-parameter logistic function analysis. That is, the dose-response of the reference and sample defibrotide curves can be described by a 4-parameter logistic function:

$$υ = δ + \frac{α - β}{1 + {e}^{- β ( x - γ )}}$$

Where, ν is the response, a is the upper asymptote, δ is the lower asymptote, β is the slope-factor, and γ is the horizontal location of the sample curve on the x axis. The potency of the defibrotide test sample was determined by calculating a potency ratio against the defibrotide reference standard. The potency ratio for the defibrotide test sample was 1.157 (FIG. 7).

### Example 8

The present example evaluates the precision of the defibrotide potency determinations of the cell-based protection assay. The potency of the same defibrotide test sample was measured, against a defibrotide reference standard, in repeated assays by different analysts, using different batches of qualified medium, cell batches and pipetting devices.

SK-HEP-1 cells were grown and expanded according to the above mentioned procedure. The cells were plated at a cell density of about 50,000 cells/ml in poly-D-lysine-coated microplates. The cytotoxic agent was fludarabine at a concentration of 10 μg/ml. The defibrotide reference standard and the defibrotide test sample were added to separate wells at the same series of concentrations as described in Example 7. Four replicate wells were run for each experimental condition. The viability of the cells was assessed with the CCK-8 assay kit after 72 hours of incubation.

The absorbances measured for each well in each assay run were subject to a 4-parameter logistic function analysis and potency ratio against the defibrotide reference standard was calculated as described in Example 7. The potency ratio of the same defibrotide test sample in each assay run is shown in Table 2. From the potency measurements by different analysts under the variable conditions shown in Table 2, the assay has a high a precision (% relative standard deviation of 7.8) and a low bias of <3%.

TABLE 2
Precision of the assay is demonstrated by analysing the same test
sample, against the defibrotide standard, in different assay runs,
on different days and cells of a different passage number.

| | | | Cell | | |
|---|---|---|---|---|---|
| Assay | | | passage | Pipetting | Measured |
| run | Day | Analyst | number | device set | Potency |
| 1 | 1 | 1 | 1 | #1 | 0.901 |
| 2 | 2 | 1 | 1 | #2 | 0.998 |
| 3 | 2 | 1 | 2 | #1 | 0.937 |
| 4 | 2 | 1 | 1 | #1 | 1.087 |
| 5 | 2 | 1 | 2 | #2 | 1.011 |
| 6 | 3 | 2 | 1 | #1 | 1.057 |
| 7 | 3 | 2 | 2 | #1 | 1.068 |
| 8 | 3 | 1 | 2 | #1 | 0.944 |
| 9 | 4 | 2 | 1 | #1 | 0.971 |
| 10 | 4 | 2 | 2 | #1 | 1.11 |
| 11 | 5 | 2 | 1 | #2 | 1.135 |
| 12 | 5 | 2 | 2 | #2 | 1.13 |

### Example 9

The present example shows a comparison of the potency as determined by the cell-based protection assay for three different batches of defibrotide and a defibrotide reference standard.

SK-HEP-1 cells were grown and expanded according to the above mentioned procedure. The cells were plated at a cell density of about 50,000 cells/ml in poly-D-lysine-coated microplates. The cytotoxic agent was fludarabine at a concentration of 10 μg/ml. The defibrotide reference standard and the different defibrotide test samples from three separate batches were added to separate wells at a concentration of 80, 40, 20, 10, 5, 2.5, or 1.25 μg/ml. Four replicate wells were run for each experimental condition. The viability of the cells was assessed with the CCK-8 assay kit after 72 hours of incubation.

The absorbances measured for each well were submitted to a 4-parameter logistic function analysis and the potency ratio for each batch of defibrotide was calculated as described in Example 7. The potency ratio relative to the defibrotide standard, of each of the defibrotide test samples from the three batches is shown in Table 3.

TABLE 3
Potency ratios for three different batches of defibrotide
analysed against a defibrotide reference standard.

| | Linearity, | | |
|---|---|---|---|
| | Regression and | 95% | |
| Defibrotide | Parallelism | Confidence | |
| batch | tests | Interval | Potency |
| 1080030021 | Passed | 0.823-0.989 | 0.902 |
| 1080060117 | Passed | 0.697-0.889 | 0.787 |
| 1080010016 | Passed | 1.116-1.725 | 1.387 |

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method of treating Veno-Occlusive Disease comprising administering to a patient in need thereof a pharmaceutical composition comprising defibrotide at a standardized potency ratio between 0.9 and 1.2 compared with a reference batch of defibrotide, wherein the potency of the defibrotide is standardized by a method comprising the steps of:
growing mammalian cells in culture;
incubating the cells with a solution containing at least one cytotoxic agent and at least one concentration of a sample batch of defibrotide;
measuring the viability of the cells after an incubation period;
comparing the cell viability of the sample batch to the cell viability for the reference batch of defibrotide; and
calculating the potency of the sample batch of defibrotide based on the comparison.

2. The method of claim 1, wherein the cells are incubated with the cytotoxic agent and at least four different concentrations of defibrotide from the sample batch, and wherein the cell viability is determined for each of the concentrations to create a dose-response curve.

3. The method of claim 1, wherein the cell viability measured for the sample batch of defibrotide is compared to a calibration curve obtained from cell viability measurements with the reference batch of defibrotide.

4. The method of claim 1, wherein calculating the potency of the defibrotide sample batch comprises determining a potency ratio relative to the potency of the reference batch of defibrotide.

5. The method of claim 1, wherein the reference batch of defibrotide has a cell protection activity of between 630 to 905 units/mg.

6. The method of claim 1, wherein the mammalian cells are human endothelial cells, human epithelial cells, human liver sinusoidal endothelial cells, or human microvascular endothelial cells.

7. The method of claim 1, wherein the mammalian cells are present at a density of about 5×10⁴ cells/ml to about 5×10⁵ cells/ml.

8. The method of claim 1, wherein the cytotoxic agent is fludarabine, 9-beta-D-arabinofuranosyl-2-fluoroadenine (F-Ara-A), or doxorubicin.

9. The method of claim 8, wherein fludarabine or F-Ara-A is present in the solution at a concentration of about 10 μg/ml to about 50 μg/ml or wherein doxorubicin is present in the solution at a concentration of about 0.1 μg/ml to about 10 μg/ml.

10. The method of claim 1, wherein the at least one concentration of defibrotide from the sample batch is in the range of about 1 μg/ml to about 100 μg/ml.

11. The method of claim 1, wherein the incubation period is at least 24 hours, at least 48 hours, or at least 72 hours.

12. The method of claim 1, wherein measuring cell viability comprises performing a colorimetric assay based on the reduction of tetrazolium dyes.

13. The method of claim 12, wherein the tetrazolium dye is 3-(4,5-dimethylthiazol-2-yl)-2,5-diphenyltetrazolium bromide or 2-(2-methoxy-4-nitrophenyl)-3-(4-nitrophenyl)-5-(2,4-disulfophenyl)-2H-tetrazolium.

14. The method of claim 1, wherein measuring cell viability comprises measuring the absorbance of the solution following incubation with the cells.

15. The method of claim 1, wherein the sample batch of defibrotide is extracted from bovine tissue or porcine tissue.

16. A method of treating Veno-Occlusive Disease comprising administering to a patient in need thereof a pharmaceutical composition comprising defibrotide at a standardized potency ratio between 0.9 and 1.2 compared with a reference batch of defibrotide, wherein the potency of the defibrotide is standardized by a method comprising the steps of:
growing mammalian cells in culture in a multi-well microtiter plate;
incubating the cells with a solution containing at least one cytotoxic agent and at least four different concentrations of defibrotide from a sample batch;
collecting dose response data points simultaneously;
measuring the viability of the cells after an incubation period;
comparing the cell viability for the at least four concentrations of the sample batch to the cell viability for the reference batch of defibrotide; and
calculating the potency of the sample batch of defibrotide based on the comparison.

17. The method of claim 16, wherein the cells are incubated with a solution containing defibrotide at a concentration between 1.25 to 80 μg/mL.

18. The method of claim 16, wherein the cell viability is determined for each of the concentrations to create a dose-response curve.

19. The method of claim 16, wherein the cytotoxic agent fludarabine or F-Ara-A is present in the solution at a concentration of about 10 μg/ml.

20. The method of claim 16, wherein the multi-well microtiter plate is a 96-well plate.

21. The method of claim 16, wherein at least 3 replicates of incubating the cells with a solution containing at least one cytotoxic agent and at least four different concentrations of defibrotide from the sample batch is performed.
